(19) United States
(12) Patent Application Publication
(10) Pub. No.: US 2026/0076890 A1
(43) Pub. Date: Mar. 19, 2026

(54) AQUEOUS PERSONAL CARE RINSE OFF COMPOSITION

(71) Applicants: Dow Global Technologies LLC, Midland, MI (US); Dow Silicones Corporation, Midland, MI (US)

(72) Inventors: Tanner McDaniel, Midland, MI (US); Timothy J. Young, Bay City, MI (US); Sung-Yu Ku, Manvel, TX (US); Wanglin Yu, Pearland, TX (US); Janet McMillan, Bay City, MI (US); Bethany K. Johnson, Midland, MI (US); Isabelle Van Reeth, Houdeng Goegnies (BE)

(21) Appl. No.: 19/108,591

(22) PCT Filed: Sep. 13, 2023
(86) PCT No.: PCT/US2023/032571

§ 371 Date: Mar. 4, 2025

Related U.S. Application Data
(60) Provisional application No. 63/408,129, filed on Sep. 20, 2022.

(51) Int. Cl. A61K 8/86; A61K 8/20; A61K 8/44; A61K 8/60; A61Q 5/02; A61Q 5/12; A61Q 19/10

(52) U.S. Cl. CPC A61K 8/86; A61K 8/20; A61K 8/442; A61K 8/604; A61Q 5/02; A61Q 5/12; A61Q 19/10; A61K 2800/48; A61K 2800/805

(57) ABSTRACT

An aqueous personal care rinse off composition is provided, comprising: a dermatologically acceptable aqueous vehicle; and a dermatologically acceptable cleaning surfactant, wherein the dermatologically acceptable cleaning surfactant comprises an alcohol ethoxysulfate surfactant of formula (I) wherein each R¹ and R² is independently a C₁₋₁₆ alkyl group; wherein the sum of the carbon atoms in R¹ and R² is 7 to 17; wherein M⁺ is a cation balancing the negative charge of the —SO₃— anion of formula (I); and wherein n is 1 in 95 to 100 mol % of the alcohol ethoxysulfate surfactant of formula (I).

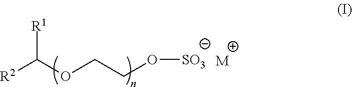

The present invention relates to a personal care rinse off composition. In particular, the present invention relates to a personal care rinse off composition containing: a dermatologically acceptable aqueous vehicle; and a dermatologically acceptable cleaning surfactant, wherein the dermatologically acceptable cleaning surfactant comprises an alcohol ethoxysulfate surfactant of formula I

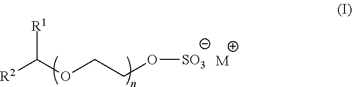

wherein each R¹ and R² is independently a C₁₋₁₆ alkyl group; wherein the sum of the carbon atoms in R¹ and R² is 7 to 17; wherein M⁺ is a cation balancing the negative charge of the —SO₃⁻ anion in formula I; and wherein n is 1 in 95 to 100 mol % of the alcohol ethoxysulfate surfactant of formula I.

Alkyl ethoxy sulfate anionic surfactants (e.g., alcohol ethoxysulfate surfactants) have established use in a variety of personal care cleansing formulations, such as shampoos and body wash formulations. Conventional AES anionic surfactants; however, have been associated with undesirable 1,4 dioxane content. Regulators are increasingly restricting the amount of 1,4 dioxane that may be present in consumer products. For example, New York State has banned all but trace amounts of 1,4 dioxane in cosmetics, personal care, and cleaning products. Typically, a consumer product must contain less than 10 parts per million by weight (ppm) of 1,4 dioxane to be compliant with the regulations. One contributor of the unintended incorporation of 1,4 dioxane in consumer products can be the inclusion of alkyl ethoxy sulfate anionic surfactants.

Inclusion of 1,4 dioxane in conventional AES surfactants is believed to occur at multiple points in time. A first point of 1,4 dioxane generation in conventional AES surfactants is believed to occur during the sulfation process of alcohol ethoxylates to make alcohol ethoxy sulfates. The alcohol ethoxylate intermediates for the production of conventional alcohol ethoxy sulfate surfactants are made via ethoxylation (i.e., the reaction of an alcohol with ethylene oxide) that typically results in a distribution of alcohol ethoxylate oligomers. It is believed that under the sulfation process conditions during the manufacture of conventional AES surfactants, 1,4 dioxane can form. A second point of 1,4 dioxane generation in association with conventional AES surfactants is believed to occur during handling and processing of the conventional AES surfactants. Handling and processing of conventional AES surfactants often involves acidic conditions at ambient, or elevated, temperature. Prolonged exposure to acidic environments for conventional AES surfactants and their alcohol ethoxylate precursors may result in the formation of 1,4 dioxane. Further, exposure to elevated temperatures (e.g., up to 280° C.) during processing, storage and/or handling may result in the decomposition of conventional AES surfactants resulting in the formation of dioxane.

Traditionally, 1,4 dioxane content in conventional AES surfactants and products incorporating such surfactants has been addressed by the use of stripping techniques. For example, where 1,4 dioxane concentrations are above a target threshold, a stripping process is employed to remove excess 1,4 dioxane from the conventional AES surfactants or product incorporating same. The stripping process is not only expensive and time consuming, but also is not a guarantee to meet the increasingly stringent regulatory requirements. Further, as 1,4 dioxane may form over time as a response to how the conventional AES surfactant or product is handled and further processed, any previously applied stripping techniques may be nullified by the generation of new 1,4 dioxane. As such, ensuring that a product comprising an AES surfactant is compliant with the appropriate regulations by the time it is sold to an end consumer is a difficult challenge.

Accordingly, there remains a need for personal care cleansing formulations having an alcohol ethoxysulfate surfactant that resists forming 1,4 dioxane both during the sulfation process to form the surfactant and subsequently when the alcohol ethoxysulfate surfactant is exposed to elevated temperatures of up to 280° C.

The present invention provides an aqueous personal care rinse off composition, comprising: a dermatologically acceptable aqueous vehicle; and a dermatologically acceptable cleaning surfactant, wherein the dermatologically acceptable cleaning surfactant comprises an alcohol ethoxysulfate surfactant of formula I

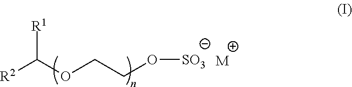

wherein each R¹ and R² is independently a C₁₋₁₆ alkyl group; wherein the sum of the carbon atoms in R¹ and R² is 7 to 17; wherein M⁺ is a cation balancing the negative charge of the —SO₃⁻ anion in formula I; and wherein n is 1 in 95 to 100 mol % of the alcohol ethoxysulfate surfactant of formula I.

The present invention provides an aqueous personal care rinse off composition, comprising: a dermatologically acceptable aqueous vehicle; and a dermatologically acceptable cleaning surfactant, wherein the dermatologically acceptable cleaning surfactant comprises an alcohol ethoxysulfate surfactant of formula I; wherein each R¹ and R² is independently a C₁₋₁₆ alkyl group; wherein the sum of the carbon atoms in R¹ and R² is 7 to 17; wherein M⁺ is a cation balancing the negative charge of the —SO₃⁻ anion in formula 1; wherein n is 1 in 95 to 100 mol % of the alcohol ethoxysulfate surfactant of formula I; and wherein the alcohol ethoxysulfate surfactant of formula I contains <9 ppm of 1,4-dioxane.

The present invention provides an aqueous personal care rinse off composition, comprising: a dermatologically acceptable aqueous vehicle; and a dermatologically acceptable cleaning surfactant, wherein the dermatologically acceptable cleaning surfactant comprises an alcohol ethoxysulfate surfactant of formula I; wherein each R¹ and R² is independently a C₁₋₁₆ alkyl group; wherein the sum of the carbon atoms in R¹ and R² is 7 to 17; wherein M⁺ is a cation balancing the negative charge of the —SO₃⁻ anion in formula 1; wherein n is 1 in 95 to 100 mol % of the alcohol ethoxysulfate surfactant of formula I; wherein the alcohol ethoxysulfate surfactant of formula I contains <9 ppm of 1,4-dioxane; and wherein the alcohol ethoxysulfate surfactant of formula I is resistant to forming 1,4-dioxane when exposed to a temperature of up to 280° C.

The present invention provides an aqueous personal care rinse off composition, comprising: a dermatologically acceptable aqueous vehicle; and a dermatologically acceptable cleaning surfactant, wherein the dermatologically acceptable cleaning surfactant comprises an alcohol ethoxysulfate surfactant of formula I; wherein each R¹ and R² is independently a C₁₋₁₆ alkyl group; wherein the sum of the carbon atoms in R¹ and R² is 7 to 17; wherein M⁺ is a cation balancing the negative charge of the —SO₃⁻ anion in formula I; wherein n is 1 in 95 to 100 mol % of the alcohol ethoxysulfate surfactant of formula I; and wherein the aqueous personal care rinse off composition contains <2 wt %, based on solids weight of the aqueous personal care rinse off composition, of an alcohol sulfate surfactant of formula II

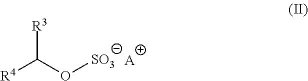

wherein each R³ and R⁴ is independently a C₁₋₁₆ alkyl group; wherein the sum of the carbon atoms is R³ and R⁴ is 7 to 17 and wherein A⁺ is a cation balancing the negative charge on the —SO₃⁻ anion in formula II.

The present invention provides an aqueous personal care rinse off composition, comprising: a dermatologically acceptable aqueous vehicle; a thickening salt and a dermatologically acceptable cleaning surfactant, wherein the dermatologically acceptable cleaning surfactant comprises an alcohol ethoxysulfate surfactant of formula I; wherein each R¹ and R² is independently a C₁₋₁₆ alkyl group; wherein the sum of the carbon atoms in R¹ and R² is 7 to 17; wherein M⁺ is a cation balancing the negative charge of the —SO₃⁻ anion in formula I; wherein n is 1 in 95 to 100 mol % of the alcohol ethoxysulfate surfactant of formula I; wherein the aqueous personal care rinse off composition contains <2 wt %, based on solids weight of the aqueous personal care rinse off composition, of an alcohol sulfate surfactant of formula II; wherein each R³ and R⁴ is independently a C₁₋₁₆ alkyl group; wherein the sum of the carbon atoms is R³ and R⁴ is 7 to 17 and wherein A⁺ is a cation balancing the negative charge on the —SO₃⁻ anion in formula II.

The present invention provides a method of cleaning at least one of mammalian skin and hair, comprising: (a) applying an aqueous personal care rinse off composition according to the present invention to the skin or hair of a mammal; and (b) rinsing the aqueous personal care rinse off composition from the skin or hair with a rinse water.

### DETAILED DESCRIPTION

We have surprisingly found that alcohol ethoxysulfate surfactant of formula I

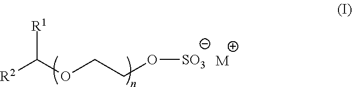

wherein each R¹ and R² is independently a C₁₋₁₆ alkyl group; wherein the sum of the carbon atoms in R¹ and R² is 7 to 17; wherein M⁺ is a cation balancing the negative charge of the —SO₃⁻ anion in formula I; and wherein n is 1 in 95 to 100 mol % of the alcohol ethoxysulfate surfactant of formula I resists forming 1,4 dioxane both during the sulfation process to form the alcohol ethoxysulfate surfactant of formula I and subsequently when the alcohol ethoxysulfate surfactant of formula I is exposed to elevated temperatures of up to 280° C. during processing, storage and/or handling.

We have also surprisingly found that alcohol ethoxysulfate surfactant of formula T

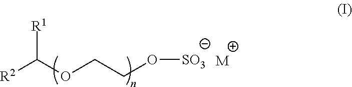

wherein each R¹ and R² is independently a C₁₋₁₆ alkyl group; wherein the sum of the carbon atoms in R¹ and R² is 7 to 17; wherein M⁺ is a cation balancing the negative charge of the —SO₃⁻ anion in formula I; and wherein n is 1 in 95 to 100 mol % of the alcohol ethoxysulfate surfactant of formula I facilitates a lower level of salt for dilution thickening of aqueous personal care rinse off compositions comprising same compared to conventional sodium laureth sulfate surfactants.

Unless otherwise indicated, ratios, percentages, parts, and the like are by weight (e.g., “ppm” means parts per million by weight).

The term “cosmetically acceptable” as used herein and in the appended claims refers to ingredients typically used in personal care compositions, and is intended to underscore that materials that are toxic when present in the amounts typically found in personal care compositions are not contemplated as part of the present invention.

The term “solids weight” as used herein and in the appended claims in reference to the aqueous personal care rinse off composition and the alcohol ethoxysulfate surfactant of formula I means dry weight, i.e., excluding any water that may be present.

Preferably, the aqueous personal care rinse off composition of the present invention is selected from the group consisting of a shampoo, a conditioning shampoo, a body wash formulation, an exfoliating body wash formulation, a facial wash formulation, an exfoliating facial wash formulation, a liquid hand soap formulation, a mild cleansing formulation and the like. More preferably, the aqueous personal care rinse off composition of the present invention is selected from the group consisting of a shampoo, a conditioning shampoo, a body wash formulation, an exfoliating body wash formulation, a facial wash formulation, an exfoliating facial wash formulation, a liquid hand soap formulation, and a mild cleansing formulation. Still more preferably, the aqueous personal care rinse off composition of the present invention is selected from the group consisting of a shampoo, a conditioning shampoo, body wash formulation, a facial wash formulation and a liquid hand soap formulation. Most preferably, the aqueous personal care rinse off composition of the present invention is a shampoo or body wash formulation.

Preferably, the aqueous personal care rinse off composition of the present invention, comprises: a dermatologically acceptable aqueous vehicle (preferably, 25 to 99 wt % (more preferably, 30 to 95 wt %; still more preferably, 40 to 92 wt %; most preferably, 70 to 90 wt %), based on weight of the aqueous personal care rinse off composition, of the dermatologically acceptable aqueous vehicle); and a dermatologically acceptable cleaning surfactant (preferably, 0.5 to 60 wt % (more preferably, 1 to 50 wt %; still more preferably, 5 to 30 wt %; most preferably, 7 to 20 wt %), based on weight of the aqueous personal care rinse off composition, of the dermatologically acceptable aqueous vehicle), wherein the dermatologically acceptable cleaning surfactant comprises an alcohol ethoxysulfate surfactant of formula I

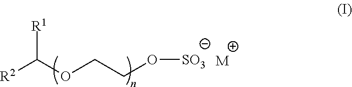

wherein each R¹ and R² is independently a C₁₆ alkyl group (preferably, a C₁₋₁₅ alkyl group; more preferably, a C₁₋₁₄ alkyl group; most preferably, a linear C₁₋₁₃); wherein the sum of the carbon atoms in R¹ and R² is 7 to 17 (preferably, 10 to 16; more preferably, 11 to 15; most preferably, 12 to 14)(preferably, wherein R¹ and R² are linear alkyl groups); wherein M⁺ is a cation balancing the negative charge of the —SO₃⁻ anion in formula I (preferably, wherein M⁺ is a cation selected from the group consisting of a nitrogen containing cation (e.g., an ammonium cation), a metal cation (e.g., an alkali metal cation, an alkaline earth metal cation), a boron containing cation and a phosphorous containing cation; more preferably, an ammonium cation, an alkali metal cation and an alkaline earth metal cation; still more preferably, an ammonium cation, a sodium cation and a calcium cation; most preferably, a sodium cation); and wherein n is 1 in 95 to 100 mol % (preferably, 96 to 100 mol %; more preferably, 97 to 100 mol %; most preferably, 97.5 to 100 mol %) of the alcohol ethoxysulfate surfactant of formula I (preferably, as determined using ¹³C nuclear magnetic resonance characterization).

Preferably, the personal care rinse off composition of the present invention, contains 25 to 99 wt % (preferably, 30 to 95 wt %; more preferably, 10 to 92 wt %; most preferably, 70 to 90 wt %), based on weight of the personal care composition, of a dermatologically acceptable vehicle. More preferably, the personal care rinse off composition of the present invention, contains 25 to 99 wt % (preferably, 30 to 95 wt %; more preferably, 10 to 92 wt %; most preferably, 70 to 90 wt %), based on weight of the personal care composition, of a dermatologically acceptable vehicle; wherein the dermatologically acceptable aqueous vehicle is selected from the group consisting of water, and a mixture of water with a C₁₋₄ alcohol. Still more preferably, the personal care rinse off composition of the present invention, contains 25 to 99 wt % (preferably, 30 to 95 wt %; more preferably, 10 to 92 wt %; most preferably, 70 to 90 wt %), based on weight of the personal care composition, of a dermatologically acceptable vehicle; wherein the dermatologically acceptable vehicle comprises water. Most preferably, the personal care rinse off composition of the present invention, contains 25 to 99 wt % (preferably, 30 to 95 wt %; more preferably, 10 to 92 wt %; most preferably, 70 to 90 wt %), based on weight of the personal care composition, of a dermatologically acceptable vehicle; wherein the dermatologically acceptable vehicle is water.

Preferably, the aqueous personal care rinse off composition of the present invention, comprises 0.5 to 60 wt % (preferably, 1 to 50 wt %; more preferably, 5 to 30 wt %; most preferably, 7 to 20 wt %), based on weight of the aqueous personal care rinse off composition, of a dermatologically acceptable cleaning surfactant, wherein the dermatologically acceptable cleaning surfactant comprises an alcohol ethoxysulfate surfactant of formula I

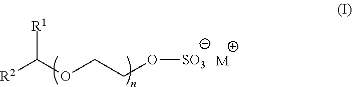

wherein each R¹ and R² is independently a C₁₋₁₆ alkyl group (preferably, a C₁₋₁₅ alkyl group; more preferably, a C₁₋₁₄ alkyl group; most preferably, a linear C₁₋₁₃); wherein the sum of the carbon atoms in R¹ and R² is 7 to 17 (preferably, 10 to 16; more preferably, 11 to 15; most preferably, 12 to 14)(preferably, wherein R¹ and R² are linear alkyl groups); wherein M⁺ is a cation balancing the negative charge of the —SO₃⁻ anion in formula I (preferably, wherein M⁺ is a cation selected from the group consisting of a nitrogen containing cation (e.g., an ammonium cation), a metal cation (e.g., an alkali metal cation, an alkaline earth metal cation), a boron containing cation and a phosphorous containing cation; more preferably, an ammonium cation, an alkali metal cation and an alkaline earth metal cation; still more preferably, an ammonium cation, a sodium cation and a calcium cation; most preferably, a sodium cation); and wherein n is 1 in 95 to 100 mol % (preferably, 96 to 100 mol %; more preferably, 97 to 100 mol %; most preferably, 97.5 to 100 mol %) of the alcohol ethoxysulfate surfactant of formula I (preferably, as determined using ¹³C nuclear magnetic resonance characterization).

Preferably, the alcohol ethoxysulfate surfactant of formula I contains <9 ppm (preferably, <8 ppm; more preferably, <7 ppm; still more preferably, <6 ppm; yet more preferably, <5 ppm; still yet more preferably, <4 ppm; yet still more preferably, <3 ppm; still even more preferably, <2 ppm; yet even more preferably, <1 ppm; still yet even more preferably, <0.25 ppm; most preferably, less than the detectable limit), based on solids weight of the alcohol ethoxysulfate surfactant of formula I, of 1,4-dioxane (preferably, wherein the 1,4-dioxane content is measured by liquid injection low temperature gas chromatography-mass spectrometry method for organic layer and liquid chromatography-mass spectrometry method for aqueous layer).

Preferably, the alcohol ethoxysulfate surfactant of formula I contains <2 wt % (preferably, <1.75 wt %; more preferably, <1.5 wt %; still more preferably, <1.25 wt %; yet more preferably, <1.1 wt %; most preferably, <1 wt %), based on solids weight of the alcohol ethoxysulfate surfactant of formula I, of an alcohol sulfate surfactant of formula II

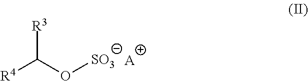

wherein each R³ and R⁴ is independently a C₁₋₁₆ alkyl group (preferably, a C₁₋₁₅ alkyl group; more preferably, a C₁₋₁₄ alkyl group; most preferably, a linear C₁₋₁₃); wherein the sum of the carbon atoms in R¹ and R² is 7 to 17 (preferably, 10 to 16; more preferably, 11 to 15; most preferably, 12 to 14)(preferably, wherein R¹ and R² are linear alkyl groups); and wherein A⁺ is a cation balancing the negative charge of the —SO₃⁻ anion in formula II (preferably, wherein A⁺ is a cation selected from the group consisting of a nitrogen containing cation (e.g., an ammonium cation), a metal cation (e.g., an alkali metal cation, an alkaline earth metal cation), a boron containing cation and a phosphorous containing cation; more preferably, an ammonium cation, an alkali metal cation and an alkaline earth metal cation; still more preferably, an ammonium cation, a sodium cation and a calcium cation; most preferably, a sodium cation).

Preferably, the personal care rinse off composition of the present invention comprises an alcohol ethoxysulfate surfactant of formula I as described above, wherein the alcohol ethoxysulfate surfactant of formula I has elevated thermal stability. More preferably, the personal care rinse off composition of the present invention comprises an alcohol ethoxysulfate surfactant of formula I as described above, wherein the alcohol ethoxysulfate surfactant of formula I has enhanced thermal stability. The term “elevated thermal stability” as used herein and in the appended claims means that the alcohol ethoxysulfate surfactant of formula I when heated to 110° C. contains <9 ppm (preferably, <8 ppm; more preferably, <7 ppm; still more preferably, <6 ppm; yet more preferably, <5 ppm; still yet more preferably, <4 ppm; yet still more preferably, <3 ppm; still even more preferably, <2 ppm; yet even more preferably, <1 ppm; most preferably, <0.5 ppm), based on solids weight of the alcohol ethoxysulfate surfactant of formula I, of 1,4-dioxane (preferably, wherein the 1,4-dioxane content is measured by liquid injection low temperature gas chromatography-mass spectrometry method for organic layer and liquid chromatography-mass spectrometry method for aqueous layer). The term “enhanced thermal stability” as used herein and in the appended claims means that the alcohol ethoxysulfate surfactant of formula I when heated to 280° C. contains <10 ppm, based on solids weight of the alcohol ethoxysulfate surfactant of formula I, of 1,4-dioxane (preferably, wherein the 1,4-dioxane content is measured by liquid injection gas chromatography-mass spectrometry method for organic layer and liquid chromatography-mass spectrometry method for aqueous layer).

Preferably, the aqueous personal care rinse off composition of the present invention, comprises 0.5 to 60 wt % (preferably, 1 to 50 wt %; more preferably, 5 to 30 wt %; most preferably, 7 to 20 wt %), based on weight of the aqueous personal care rinse off composition, of a dermatologically acceptable cleaning surfactant; wherein the dermatologically acceptable cleaning surfactant comprises an alcohol ethoxysulfate surfactant of formula I and at least one additional surfactant; wherein the weight ratio of the alcohol ethoxysulfate surfactant of formula I to the at least one additional surfactant in the aqueous personal care rinse off composition is 10:1 to 1:10 (preferably, 7:1 to 1:7; more preferably, 5:1 to 1:1; most preferably, 3.5:1 to 1.75:1). More preferably, the aqueous personal care rinse off composition of the present invention, comprises 0.5 to 60 wt % (preferably, 1 to 50 wt %; more preferably, 5 to 30 wt %; most preferably, 7 to 20 wt %), based on weight of the aqueous personal care rinse off composition, of a dermatologically acceptable cleaning surfactant; wherein the dermatologically acceptable cleaning surfactant comprises an alcohol ethoxysulfate surfactant of formula I and at least one additional surfactant; wherein the at least one additional surfactant is selected from the group consisting of alkyl polyglucosides (e.g., lauryl glucoside, cocoyl-glucoside, decyl glucoside), alkyl poly pentosides (e.g., C₅₋₁₈ glycosides), glycinates (e.g., sodium cocoyl glycinate), betaines (e.g., alkyl betaines such as cetyl betaine and amido betaines such as cocamidopropyl betaine), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), amphoacetates (e.g., sodium cocoamphoacetate), sulfonates (e.g., sodium C₁₄₋₁₆ olefin sulfonate), sulfates (e.g., sodium lauryl sulfate), succinates (e.g., disodium lauryl sulfosuccinate), sophorolipids (e.g., lactone form, acid form), rhamnolipids and mixtures thereof; and wherein the weight ratio of alcohol ethoxysulfate surfactant of formula I to the at least one additional surfactant in the aqueous personal care rinse off composition is 10:1 to 1:10 (preferably, 7:1 to 1:7; more preferably, 5:1 to 1:1; most preferably, 3.5:1 to 1.75:1). More preferably, the aqueous personal care rinse off composition of the present invention, comprises 0.5 to 60 wt % (preferably, 1 to 50 wt %; more preferably, 5 to 30 wt %; most preferably, 7 to 20 wt %), based on weight of the aqueous personal care rinse off composition, of a dermatologically acceptable cleaning surfactant; wherein the dermatologically acceptable cleaning surfactant comprises an alcohol ethoxysulfate surfactant of formula I and at least one additional surfactant; wherein the at least one additional surfactant is selected from the group consisting of a sulfate, a sophorolipid, a rhamnolipid, a betaine, a glycinate, a glucoside and a succinate; and wherein the weight ratio of alcohol ethoxysulfate surfactant of formula I to the at least one additional surfactant in the aqueous personal care rinse off composition is 10:1 to 1:10 (preferably, 7:1 to 1:7; more preferably, 5:1 to 1:1; most preferably, 3.5:1 to 1.75:1). Still more preferably, the aqueous personal care rinse off composition of the present invention, comprises 0.5 to 60 wt % (preferably, 1 to 50 wt %; more preferably, 5 to 30 wt %; most preferably, 7 to 20 wt %), based on weight of the aqueous personal care rinse off composition, of a dermatologically acceptable cleaning surfactant; wherein the dermatologically acceptable cleaning surfactant comprises an alcohol ethoxysulfate surfactant of formula I and at least one additional surfactant; wherein the at least one additional surfactant is selected from the group consisting of alkyl polyglucosides (e.g., lauryl glucoside, cocoyl-glucoside, decyl glucoside) and betaines; and wherein the weight ratio of alcohol ethoxysulfate surfactant of formula I to the at least one additional surfactant in the aqueous personal care rinse off composition is 10:1 to 1:10 (preferably, 7:1 to 1:7; more preferably, 5:1 to 1:1; most preferably, 3.5:1 to 1.75:1). Most preferably, the aqueous personal care rinse off composition of the present invention, comprises 0.5 to 60 wt % (preferably, 1 to 50 wt %; more preferably, 5 to 30 wt %; most preferably, 7 to 20 wt %), based on weight of the aqueous personal care rinse off composition, of a dermatologically acceptable cleaning surfactant; wherein the dermatologically acceptable cleaning surfactant comprises an alcohol ethoxysulfate surfactant of formula I and at least one additional surfactant; wherein the at least one additional surfactant is a mixture of an alkyl polyglucoside and a cocamidopropyl betaine; and wherein the weight ratio of alcohol ethoxysulfate surfactant of formula I to the at least one additional surfactant in the aqueous personal care rinse off composition is 10:1 to 1:10 (preferably, 7:1 to 1:7; more preferably, 5:1 to 1:1; most preferably, 3.5:1 to 1.75:1).

Preferably, the personal care rinse off composition of the present invention, further comprises at least one optional personal care ingredient. More preferably, the personal care rinse off composition of the present invention, further comprises at least one optional personal care ingredient, wherein the at least one optional personal care ingredient is selected from the group consisting of emollients (e.g., hydrocarbon oils, esters, natural oils), cosmetically acceptable silicones (e.g., amodimethicone, cyclomethicone, dimethicone, dimethiconol, hexadecyl methicone, hexamethyldisiloxane, methicone, phenyl dimethicone, stearoxy dimethicone), waxes, soaps, sensory modifiers, lubricants, preservatives (e.g., benzoic acid, sorbic acid, phenoxyethanol), antioxidants (e.g., butylated hydroxytoluene), chelating agents, antimicrobials, pH adjusting agents/buffers/neutralizing agents, humectants (e.g., glycerin, sorbitol, monoglycerides, lecithins, glycolipids, fatty alcohols, fatty acids, polysaccharides, sorbitan esters, polysorbates (e.g., Polysorbate 20, Polysorbate 40, Polysorbate 60, and Polysorbate 80), diols (e.g., propylene glycol), diol analogs, triols, triol analogs, polymeric polyols), sunscreen actives, vitamins, proteins/amino acids, plant extracts, natural ingredients, bio-actives, fragrances/perfumes, penetrants, polymers/resins/hair fixatives/film formers, surfactants/detergents/emulsifiers/opacifying agents, volatiles/propellants/solvents/carriers, liquid vehicles/solvents/carriers, salts, anti-static agents, anti-frizz agents, antidandruff agents, hair waving/straightening agents, absorbents, colorants, hard particles, rheology modifiers (e.g., carbomer, acrylates, celluloses, stearates, natural gums) and conditioning agents (e.g., polyquaternium 10, cationic guar).

Preferably, the personal care rinse off composition of the present invention, further comprises a thickening salt. More preferably, the personal care rinse off composition of the present invention, further comprises 0.1 to 20 wt % (preferably, 0.2 to 15 wt %; more preferably, 0.3 to 10 wt %; most preferably, 0.5 to 3 wt %), based on weight of the personal care rinse off composition, of a thickening salt, the personal care rinse off composition of the present invention, further comprises 0.1 to 20 wt % (preferably, 0.2 to 15 wt %; more preferably, 0.3 to 10 wt %; most preferably, 0.5 to 3 wt %), based on weight of the personal care rinse off composition, of a thickening salt; wherein the thickening salt is selected from the group consisting of sodium chloride, potassium chloride, sodium sulfate, potassium sulfate, magnesium chloride, magnesium sulphate, zinc sulphate, ammonium chloride and monoethanolamine chloride and mixtures thereof. Still more preferably, the personal care rinse off composition of the present invention, further comprises 0.1 to 20 wt % (preferably, 0.2 to 15 wt %; more preferably, 0.3 to 10 wt %; most preferably, 0.5 to 3 wt %), based on weight of the personal care rinse off composition, of a thickening salt, the personal care rinse off composition of the present invention, further comprises 0.1 to 20 wt % (preferably, 0.2 to 15 wt %; more preferably, 0.3 to 10 wt %; most preferably, 0.5 to 3 wt %), based on weight of the personal care rinse off composition, of a thickening salt; wherein the thickening salt is selected from the group consisting of sodium chloride, potassium chloride, magnesium chloride and mixtures thereof. Most preferably, the personal care rinse off composition of the present invention, further comprises 0.1 to 20 wt % (preferably, 0.2 to 15 wt %; more preferably, 0.3 to 10 wt %; most preferably, 0.5 to 3 wt %), based on weight of the personal care rinse off composition, of a thickening salt, the personal care rinse off composition of the present invention, further comprises 2 to 35 wt % (preferably, 4 to 20 wt %; more preferably, 5 to 15 wt %; most preferably, 6 to 10 wt %), based on weight of the personal care rinse off composition, of a thickening salt; wherein the thickening salt is sodium chloride.

Preferably, the personal care rinse off composition of the present invention contains <9 ppm (preferably, <5 ppm; more preferably, <4 ppm; yet more preferably, <3 ppm; still more preferably, <2 ppm; yet still more preferably, <1 ppm; still yet more preferably, <0.5 ppm; even more preferably, <0.25 ppm; still even more preferably, <0.15 ppm; most preferably, less than the detectable limit), based on solids weight of the personal care rinse off composition, of 1,4-dioxane (preferably, wherein the 1,4-dioxane content is measured by liquid injection low temperature gas chromatography-mass spectrometry method for organic layer and liquid chromatography-mass spectrometry method for aqueous layer).

Preferably, aqueous personal care rinse off composition of the present invention, comprises <1 wt % (preferably, <0.1; more preferably, <0.01 wt %; still more preferably, <0.0015 wt %; yet more preferably, <0.001 wt %; most preferably, less than detectable limit), based on solids weight of the personal care rinse off composition, of an alcohol sulfate surfactant of formula II

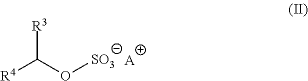

wherein each R³ and R⁴ is independently a C₁₋₁₆ alkyl group (preferably, a C₁₋₁₅ alkyl group; more preferably, a C₁₋₁₄ alkyl group; most preferably, a linear C₁₋₁₃); wherein the sum of the carbon atoms in R¹ and R² is 7 to 17 (preferably, 10 to 16; more preferably, 11 to 15; most preferably, 12 to 14)(preferably, wherein R¹ and R² are linear alkyl groups); and wherein A⁺ is a cation balancing the negative charge of the —SO₃⁻ anion in formula II (preferably, wherein A⁺ is a cation selected from the group consisting of a nitrogen containing cation (e.g., an ammonium cation), a metal cation (e.g., an alkali metal cation, an alkaline earth metal cation), a boron containing cation and a phosphorous containing cation; more preferably, an ammonium cation, an alkali metal cation and an alkaline earth metal cation; still more preferably, an ammonium cation, a sodium cation and a calcium cation; most preferably, a sodium cation).

Preferably, the personal care rinse off composition of the present invention has a viscosity of ≥3,000 cP as determined according to the method used herein in the Examples. More preferably, the personal care rinse off composition of the present invention has a viscosity of 3,000 to 15,000 CP as determined according to the method used herein in the Examples. Still more preferably, the personal care rinse off composition of the present invention has a viscosity of 4,000 to 12,000 cP as determined according to the method used herein in the Examples. Most preferably, the personal care rinse off composition of the present invention has a viscosity of 5,000 to 9,000 cP as determined according to the method used herein in the Examples.

Preferably, the personal care rinse off composition of the present invention further comprises a pH adjusting agent. More preferably, the personal care rinse off composition of the present invention, further comprises a pH adjusting agent, wherein the personal care rinse off composition has a pH of 5 to 9 (preferably, 6 to 8; most preferably, 6.25 to 7.75).

Preferably, the pH adjusting agent is selected from the group consisting of citric acid, lactic acid, hydrochloric acid, aminoethyl propanediol, triethanolamine, monoethanolamine, sodium hydroxide, potassium hydroxide, amino-2-methyl-1-propanol. More preferably, the pH adjusting agent is selected from the group consisting of citric acid, lactic acid, sodium hydroxide, potassium hydroxide, triethanolamine, amino-2-methyl-1-propanol. Most preferably, the pH adjusting agent is selected from the group consisting of citric acid and sodium hydroxide.

Preferably, the method of cleaning at least one of mammalian skin and hair (preferably, at least one of human skin and hair) of the present invention, comprises: applying an aqueous personal care rinse off composition of the present invention to the skin or hair of a mammal (preferably of a human); and rinsing the aqueous personal care rinse off composition from the skin or hair with a rinse water.

Some embodiments of the present invention will now be described in detail in the following Examples.

### Experimental Materials

| Material | Description |
|---|---|
| Catalyst | metallosilicate catalysts defined by BEA structure and having silica to |
| | alumina ratio of 25:1 and surface area of 680 m²/g, available as |
| | CP814E from ZEOLYST INTERNATIONAL ™ of Conshohocken, PA |
| 1-Dodecene | alpha olefin available as NEODENE ™ 12 from SHELL ™ group of |
| | The Hague, Netherlands |
| 1-Tetradecene | alpha olefin available as NEODENE ™ 14 from SHELL ™ group of |
| | The Hague, Netherlands |
| Monoethylene | liquid anhydrous ethylene glycol from SIGMA ALDRICH ™ having |
| glycol | CAS number of 107-21-1 |
| ALEO1 | linear primary C₁₂₋₁₄ alcohol ethoxylate with an average of 1 moles of |
| | ethylene oxide per molecule as measured according to ¹³C NMR |
| | characterization available as SURFONIC ™ L24-1 from Huntsman |
| | Corporation, The Woodlands, Texas |
| Sulfuric acid | ACS reagent (95-98%) from Sigma Aldrich having CAS number |
| | 7664-93-9 |
| Chlorosulfonic | liquid from Sigma Aldrich having CAS number 7790-94-5 |
| acid | |
| Sodium | solid from Sigma Aldrich having CAS number 1310-73-2 |
| hydroxide | |
| (NaOH) | |
| Dichloromethane | liquid anhydrous dichloromethane from Sigma Aldrich having CAS |
| (DCM) | number 75-09-2 |
| SA3EO | secondary alcohol ethoxylate with an average of 3 moles of ethylene |
| | oxide per molecule sulfated in manner described below in connection |
| | with C₁₂EO sulfate. The base starting material is a C₁₂₋₁₄ secondary |
| | alcohol ethoxylate having a CAS number 84133-50-6 available as |
| | TERGITOL ™ 15-S-3 from The Dow Chemical Company, Midland, |
| | Michigan |
| Sodium dioctyl | solid (SDOSS) BioXtra ≥99.0% from Sigma Aldrich having CAS |
| sulfosuccinate | number 577-11-7 |
| Sodium dodecyl | dust-free pellets (SDS) ≥99.0% (GC) from Sigma Aldrich having CAS |
| sulfate | number 151-21-3 |
| Hyamine 1622 | solid benzethonium chloride ≥99.0% (AT) from Sigma Aldrich having |
| | CAS number 121-54-0 |
| Methylene Blue | a solid (certified biological stain) from Fischer Scientific having CAS |
| | number 7220-79-3 |
| Sodium sulfate | anhydrous solid (granular/certified ACS) from Fischer Scientific |
| | having CAS number 7757-82-6 |
| Ethal ® LA-4 | INCI: Laureth-4. Nonionic linear alcohol ethoxylate from Ethox |
| | Chemicals. |
| Ethal ® LA-7 | INCI: Laureth-7. Nonionic linear alcohol ethoxylate from Ethox |
| | Chemicals. |
| Polystep ® B-N-5 | Sodium lauryl sulfate from Stepan Company |

### Synthesis S1: C₁₂EO

A 3 liter (L) 3-neck glass round bottom flask, equipped with an overhead stirred through the center neck, reflux condenser and a heating jacket was used for the etherification of 1-dodecene and monoethylene glycol with the catalyst. To ensure good mixing, a pitch blade impeller was used for agitation. A reaction mixture of 551.7 grams (g) ethylene glycol and 505.8 g 1-dodecene was prepared and loaded in the reactor together with 61-g catalyst in powdered form at 23° C. The impeller stirring rate was set to be at 400 revolutions per minute (“rpm”). The reactor was heated to 135° C. in over the course of 30 minutes, held at 135° C. for 18 hours and then the reactor was cooled down to 23° C. by shutting off the heater. The reaction mixture was separated into a monoethylene glycol and catalyst phase and an olefin phase using a separation funnel.

A distillation apparatus was constructed using a 1-liter round bottom flask connected to a short path distillation head with a thermometer adapter and a condenser with a vacuum adapter at the outlet. The distillation flask was heated in an aluminum block by an IKA heated stir-plate. The distillation pot was charged with the combined olefin phase and then stirring and vacuum were applied. Significant boiling was observed but no condensate was observed or collected. The temperature of the heating block was raised to 75° C. and unreacted dodecane was collected at a distillation head temperature of 25° C. to 50° C. and a pressure of 13.3-40 pascals (Pa). The heating block temperature was raised gradually to 140° C. and an intermediate fraction containing both monoether alcohol ethoxylates and dodecenes was recovered while the head temperature increased from 50° C. to 75° C. at a pressure of 13 Pa. The C₁₂EO was collected at a head temperature of 70° C. to 115° C. and a pressure of 6 Pa to 33 Pa. The heating block temperature was raised gradually to 200° C. and an intermediate fraction containing both monoether alcohol ethoxylates and diether was collected while the head temperature increased from 115° C. to 130° C. at a pressure of 6 Pa. The distillation was discontinued and the diether, which remained in the pot, was collected. The C₁₂EO was carried to the next sulfation process to make sulfate anionic surfactant.

### Synthesis S2: C₁₄EO

A 300 mL Parr reactor with a heating jacket and controller was used for the etherification of 1-tetradecene and monoethylene glycol with a catalyst. To ensure good mixing, a pitch blade impeller was used for agitation.

The reaction mixture of 100.0 g monoethylene glycol and 100.0 g 1-tetradecene was prepared and loaded in the reactor together with 10.0 g powder form catalyst at 23° C. The impeller stirring rate was set to be at least 600 rpm. The reactor was heated up to 135° C. in 30 minutes, held at 135° C. for 6 hours and then the reactor was cooled down to room temperature by shutting off the heater. The reaction mixture was separated by a separation funnel. The reaction mixture was separated into a monoethylene glycol and catalyst phase and an olefin phase using a separation funnel. Fifteen batches were generated and the olefin phases were collected and combined for distillation.

The same distillation apparatus as used in the Synthesis S1 was used for distillation of the C₁₄EO. The distillation pot was charged with the products in the olefin phase from multiple batch reactor runs and then stirring and vacuum were applied. Significant boiling was observed but no condensate was observed or collected. The temperature of the heating block was raised to 95° C. and unreacted 1-tetradecene was collected at a distillation head temperature of 30° C. to 60° C. at a pressure of 27 Pa to 5 Pa. The heating block temperature was raised gradually to 170° C. and an intermediate fraction containing both monoether and tetradecene was recovered while the head temperature increased from 60° C. to 85° C. at a pressure of 7 Pa to 5 Pa. The C₁₄EO was collected at a head temperature of 80° C. to 115° C. and a pressure of 8 Pa to 5 Pa. The distillation was discontinued when no more material would distill over with the pot temperature set at 170° C.

### Synthesis S3: C₁₂EO Sulfate

All chemical manipulations were conducted under a dry nitrogen atmosphere. Prior to the experiment all glassware was heated in a laboratory oven to remove residual water. A 2-L three-neck round bottom flask was loaded with dichloromethane (500 mL) and C₁₂EO prepared according to Synthesis S1 (40 g, 0.173 mol, 1.0 equivalents). The reaction flask was equipped with an overhead mechanical stirrer, additional funnel, and thermocouple. Next, chlorosulfonic acid (12.7 mL, 0.191 mol, 1.1 equivalents) was carefully loaded into the additional funnel. The reaction flask was then submerged into an ice-bath and allowed to cool for 20 minutes, down to 0° C. Once the reaction was cooled, chlorosulfonic acid was added to the reaction flask dropwise, at a rate of approx. 1.0 mL per minute, over approximately 20 minutes. During the addition of chlorosulfonic acid the reaction temperature did not exceed 5° C. After the addition, the reaction was allowed to react, and the temperature was kept between 0 and 5° C., for 3 hours. At this time, the reaction was neutralized by slow dropwise addition of an aqueous NaOH solution (18.0 g NaOH in 500 mL of water, 0.9 molar). The rate of addition was slow enough to not exceed 5° C. over the course of addition. The solution became basic after the addition of −300 mL of 0.9 molar NaOH solution. Dichloromethane was then carefully removed from the biphasic reaction in vacuo. During the removal of the dichloromethane, a large amount of foam was observed. Upon removal of the dichloromethane, the remaining aqueous solution was placed in a freeze drier/lyophilizer to yield the secondary alcohol ethoxylate sulfate product, C₁₂EO Sulfate, as a whiteish solid (61.9 grams).

### Synthesis S4: C₁₄EO Sulfate

All chemical manipulations were conducted under a dry nitrogen atmosphere. Prior to the experiment all glassware was heated in a laboratory oven to remove residual water. A 2-L three-neck round bottom flask was loaded with dichloromethane (500 mL) and C₁₄EO prepared according to Synthesis S2 (50 g, 0.193 mol, 1.0 equivalents). The reaction flask was equipped with an overhead mechanical stirrer, additional funnel, and thermocouple. Next, chlorosulfonic acid (14.2 mL, 0.213 mol, 1.1 equivalents) was carefully loaded into the additional funnel. The reaction flask was then submerged into an ice-bath and allowed to cool for 20 minutes, down to 0° C. Once the reaction was cooled, chlorosulfonic acid was added to the reaction flask dropwise, at a rate of approximately 1.0 mL per minute, over approximately 20 minutes. During the addition of chlorosulfonic acid the reaction temperature did not exceed 5° C. After the addition, the reaction was allowed to react and the temperature was kept between 0° C. and 5° C., for 3 hours. At this time, the reaction was neutralized by slow dropwise addition of aqueous NaOH (18.0 g in 500 mL of water, 0.9 molar). The rate of addition was slow enough to not exceed 5° C. over the course of addition. The solution became basic after the addition of about 400 mL of 0.9 molar NaOH solution. Dichloromethane was then carefully removed from the biphasic reaction in vacuo. During the removal of DCM, a large amount of foam was observed. Upon removal of DCM, the remaining aqueous solution was placed in a freeze drier/lyophilizer to give the secondary alcohol ethoxylate sulfate product (68.6 grams).

### Synthesis S5: ALEO1 Sulfate

ALEO1 sulfate was prepared from ALEO1 in the same manner as described in Synthesis S3.

### Synthesis S6: SA3EO Sulfate

SA3EO sulfate was prepared from SA3EO in the same manner as described in Synthesis S3.

### EO Distribution of Surfactants

The distribution of EO adducts in the surfactants listed in TABLE 1 was determined by NMR or UHPLC-MS as noted using the methodology set forth below with the results provided in TABLE 1.

### Nuclear Magnetic Resonance EO Distribution Characterization (NMR)

Samples of surfactant to be analyzed were prepared by dissolving the surfactant in deuterated dimethyl sulfoxide containing 0.025 M chromium (III) acetylacetonate. Nuclear magnetic resonance (¹³C NMR) spectra of the samples were then collected on a Bruker AVANCE 400 MHz spectrometer equipped with a 10 mm cryo-probe set to 25° C., with the following parameters: a 90°-pulse, inverse-gated decoupling, a 1.38 second acquisition time, and a 6.4 second recycle delay. 2048 scans were collected. The data was processed in MNOVA, and the chemical shifts were referenced to the solvent peak at 39.52 ppm. A DEPT-135 experiment was also acquired with the same parameters, but with a 2.0 second recycle delay, and 2048 acans. The ratios of different EO adducts are calculated by integrating and comparing the intensity of the ethylene oxide alcohol end groups from about 60-61 ppm, the ethylene oxide backbone groups from about 69-70 ppm, the ethylene oxide end group ether peak from about 71-72 ppm, the unreacted primary alcohol peaks from about 60-61 ppm, and the unreacted secondary alcohol peaks from about 65-66 ppm.

### Sodium Lauryl Ether Sulfate Analysis by UHPLC-MS

Ultra high performance liquid chromatography-mass spectrometry (UHPLC-MS) conditions:

| HPLC System | Waters ACQUITY ® UPLC System | | |
|---|---|---|---|
| Column | Waters BEH C18 1.7 μm 1 × 50 mm | | |
| Mobile phase A | 5 mM ammonium hydroxide in Milli-Q water | | |
| Mobile phase B | Acetronitrile | | |
| | Time | Pump A (%) | Pump B (%) |
| Gradient | 0 | 85 | 15 |
| | 1 | 50 | 50 |
| | 1.7 | 0.1 | 99.9 |
| | 3.2 | 0.1 | 99.9 |
| | 3.38 | 85 | 15 |
| | 5.2 | 85 | 15 |

| | |
|---|---|
| Flow rate | 150 μL/min |
| Injection vol. | Full loop and PLNO (loop - 1 μL) |
| Column temp | 50° C. |
| Weak wash | 50/50 methanol/water |
| Strong wash | 1:1:1 IPA:MeOH:CAN |
| MS Instrument | Waters LCT Premier TOF Mass Spectrometer with |
| | Electrospray Ionization (ESI) |
| Data system | Mass Lynx version 4.1 |
| Detection mode | Positive and negative ion |
| ESI tip voltage | +3,000 V |
| | −2,500 V |
| Cone voltage | 35 V (positive) |
| | 35 V (negative) |
| Mass range | 150-3,700 m/z; masses extracted for alcohol |
| | ethoxylates: commercial 2-mole sodium |
| | laureth sulfate, EO 2-7 and for commercial |
| | 3-mole sodium laureth |
| | sulfate, EO 6-11; |
| | alkyl sulfate masses extracted m/z |
| | 265.2, 293.2 and 321.2 |
| Scan time | 0.1 s |
| Source temp | 150° C. |
| Desolvation | 350° C. |
| temp | |
| Cone gas | N₂ at 0 L/h |
| Desolvation gas | N₂ at 700 L/h |

Procedure: The compositions containing commercial surfactants were analyzed by ultra-high performance liquid chromatography mass spectrometry (UHPLC-MS) with electrospray ionization (ESI). For the analysis, stock solutions were prepared at concentrations of 25 ppm in a 50/50 mixture of methanol/water. The alcohol ethoxylate samples were diluted 1:100 in 50/50 methanol/water in duplicate and vortexed for a few seconds. Then they were diluted 1:10 in 50/50 methanol/water to give a final dilution of 1:1,000. Ethal® LA-4 was the standard used for the commercial 1-mole sodium laureth sulfate and Ethal® LA-7 was the standard used for the commercial 3-mole sodium laureth sulfate. Calibration standards were prepared at 10 ppm, 5 ppm, 2 ppm and 1 ppm in 50/50 methanol/water.

Alkyl sulfates were diluted from the 1:1,000 preparation in 50/50 methanol/water to give a final solution of 1:20,000. POLYSTEP® B-N-5 was used as the standard for the alkyl sulfate analysis. Standards were prepared in 50/50 methanol/water at concentrations of 5 ppm, 2 ppm, 1 ppm and 0.5 ppm.

The samples were analyzed using a Waters ACQUITY® UPLC system equipped with a Waters BEH C18 1.7 μm 1×50 mm column. Mass spectrometry was conducted using a Waters LCT Premier TOF Mass Spectrometer with ESI. Measurements were conducted in both positive and negative ion mode. Each sample preparation was injected three times for analysis. The ratios of the different EO adducts were calculated by the peak area and are reported in TABLE 1.

TABLE 1

EO Adduct (i.e., n) (mol %)

| | | | |
|---|---|---|---|
| Surfactant | 0 | 1 | 2+ |

UHPLC-MS

| | | | |
|---|---|---|---|
| Surfactant A¹ | 37 | 14 | 49 |
| Surfactant C² | 25 | 10 | 65 |

NMR

| | | | |
|---|---|---|---|
| Synthesis S1: C₁₂EO | 1 | 98 | 1 |
| Synthesis S2: C₁₄EO | 1 | 98 | 1 |
| ALEO1 | 51.2 | 2 | 46.8 |

¹Commercial 1-mole sodium laureth sulfate
²Commercial 3-mole sodium laureth sulfate

At least 95 mol % of the oligomers of the products of Syntheses S4 and S5 had an n of 1 and no more than 5 mol % the oligomers have an n of ≥2. Specifically, ≥98 mol % of the oligomers of the products of Syntheses S4 and S5 had an n of 1 and ≤2 mol % of the oligomers had an n of ≥2.

### 1,4-Dioxane Content of Surfactants

The 1,4-dioxane content in the surfactants listed in TABLE 2 was determined by liquid injection low temperature gas chromatography-mass spectrometry (GC-MS) method for organic layer and liquid chromatography-mass spectrometry (LC-MS) method for aqueous layer as noted using the methodology set forth below with the results provided in TABLE 2.

Gas chromatography-Mass spectrometry (GC-MS) conditions for organic layer 1,4-dioxane measurement:

| GC system | Agilent 7890 GC system | |
|---|---|---|
| Analytical column | DB-1 30 m × 0.32 mm × 5 μm | |
| Inlet Type | Split/Splitless | |
| Inlet Pressure | 10.95 | psi |
| Total flow | 17.2 | mL/min |
| Septum purge flow | 4 | mL/min |
| Split Ratio | 10:1 | |
| Inlet Temperature | 110° | C. |
| Injection Volume | 1 | μL |
| Column flow | 1.2 mL/min, constant flow | |
| GC Oven | 35° C. hold for 2.00 min, 10° C./min to | |
| | 300° C. hold for 5.00 min | |
| GC Run Time | 33.50 | minutes |
| MS System | Agilent 5977A MSD | |
| MS Parameters | | |
| Mode | EI | |
| Transfer line temperature | 250° | C. |
| MS Source | 230° | C. |
| MS Quad | 150° | C. |
| Acquisition Mode | SIM | |
| SIM Ion | 88.00 | m/z |
| Dwell Time | 250 | |
| Mass range | 33-750 | m/z |
| Energy | 70 | eV |
| Emission Current | 34.6 | mA |

| | |
|---|---|
| Solvent delay | None |

Standards were prepared by adding dioxane in tetrahydrofuran (“THF”) and diluting down to 0.1-100 ppm.

Samples were prepared by mixing 3.3 g from the organic (DCM) layer of the crude process mixture and 6.7 g of THF, the solution was then allowed to shake for about 20 minutes. The solids were then centrifuged to the bottom and the supernatant was vialed in an autosampler vial. Spiked samples were prepared by spiking dioxane standard in THF into separate samples at 5-10 ppm.

Liquid chromatography-mass spectrometry (LC-MS) conditions for aqueous layer 1,4-dioxane content measurement:

| Instrument | Agilent 1260 Infinity Series w/6470 Triple Quad MS | | |
|---|---|---|---|
| Column | Waters Atlantis T3 3.0 × 150 mm, 3 μm | | |
| Column temp. | 30° C. | | |
| Mobile Phase | A: Water | | |
| Solvents: | B: Acetonitrile | | |
| Flow rate | 0.40 mL/min | | |
| | Time (mins) | Pump A (%) | Pump B (%) |
| LC Conditions | 0 | 95 | 5 |
| | 1 | 95 | 5 |
| | 16 | 60 | 40 |
| | 17 | 5 | 95 |
| | 22 | 5 | 95 |

| | |
|---|---|
| Run Time | 22.00 mins |
| Post Time | 8 mins |
| Injection Volume | 5 μL |
| UV wavelength | 190-600 nm |
| APCI conditions | Spray Voltage: 3500 V (+) |
| | Gas Temp.: 350° C. |
| | APCI Heater Temp: 500° C. |
| | Gas Flow: 9 L/min |
| | Nebulizer: 60 psi |
| | Fragmentor: 77 |
| MS Parameters | Mode: SIM |
| | SIM Ion: 89.2 m/z |
| | Dwell Time: 500 msec |

Samples were injected neat or diluted with water 1:4. Standards were prepared by preparing a dioxane in THF stock solution and the diluting down with water to 0.1-100 ppm.

### Calculation of Dioxane Content Relative to Solids

The ppm of dioxane content relative to solids content in the sample is calculated according to equation 1.

$$\frac{\begin{matrix}Concentration of Dioxane in Solution ( \frac{mg}{L} or ppM ) + \\ Total Reaction Volume ( L )\end{matrix}}{Theoretical Yield of product ( mg )} \times {10}^{6} \qquad ( Eq . 1 )$$

TABLE 2

| | 1,4 Dioxane (ppm) | | | | |
|---|---|---|---|---|---|
| | | | Agueous | | |
| | Organic Phase (GC) | | Phase | | |
| Sample | 110° C. | 280° C. | (LCMS) | A | B |
| Comparative | 0.9 | 259 | 0.51 | 0.71 | 9.5 |
| S5: ALEO1 sulfate | | | | | |
| Comparative | 2.0 | 1,471 | 0.48 | 1.24 | 15.3 |
| S6: SA3EO sulfate | | | | | |
| Inventive | <0.1^{a} | 0.58 | <0.1^{a} | <0.1^{a} | <1.64^{d} |
| S3: C₁₂EO sulfate | | | | | |

A 1,4-dioxane concentration in sample calculated from average concentration in both the organic phase (at 110° C.) and the aqueous phase.
B 1,4-dioxane concentration relative to solids from average concentration in both organic phase (at 110° C.) and the aqueous phase.
^{a}Below equipment detection limit of 0.1 ppm
^{b} Calculated based on detection limit of 0.1 ppm and solids present in sample

The gas chromatography results for the SA3EO sulfate surfactant indicate the secondary alcohol with an average of 3 moles of ethylene oxide per molecule contained 2 ppm of 1,4-dioxane in the organic phase at 110° C. and indicates the potential for structures having n≥2 to produce 1,4-dioxane. Interestingly, when the inlet temperature was increased to 280° C., the dioxane content of SA3EO sulfate increased from 2 ppm to 1,471 ppm 1,4-dioxane in the organic phase. This result indicates that sulfated surfactants having n≥2 may develop observable 1,4-dioxane at 110° C., but also that such surfactants may be unstable at elevated temperatures of 280° C. which could result in significant 1,4-dioxane formation. Similarly to SA3EO sulfate, gas chromatography results for the ALEO1 sulfate surfactant indicate the formation of >9 ppm of 1,4-dioxane relative to the solids at 110° C. Further, the ALEO1 sulfate surfactant also exhibited a large generation of 1,4-dioxane (259 ppm) at 280° C., suggesting that the ALEO1 sulfate surfactant lacks stability at high temperatures. The inventive C₁₂EO sulfate surfactant with ≥95 mol % having n of 1 and <5 mol % having n≥2 demonstrates a low 1,4-dioxane content. Surprisingly, the inventive C₁₂EO sulfate surfactant also demonstrates an extremely low 1,4-dioxane content and falls below the limit of detection (LOD) of the GC and LC methods. Using the LOD as a basis, this indicates that the dioxane content for the C₁₂EO sulfate is <1.6 ppm relative to solids, at 110° C. Interestingly, the inventive C₁₂EO sulfate, when the inlet temperature is increased to 280° C., the 1,4-dioxane content of the material is still less than 1 ppm (i.e., 0.58 ppm), which indicates that the inventive C₁₂EO sulfate has increased thermal stability relative to the comparative materials.

### Comparative Examples CF1-CF5 & Examples F1-F4

### Personal Care Cleansing Formulation

The personal care cleansing formulations of Comparative Examples CF1-CF5 and Examples F1-F4, as set forth in TABLE 3, were prepared on a 15 g scale by first adding the anionic surfactant followed by the cocamidopropyl betaine surfactant, then the C₈₋₁₄ alkyl polyglucoside surfactant and then the water. The formulations were heated to 60° C. until fully dissolved. The pH of the formulations was then adjusted to 5.5-6.0 using 10 wt % citric acid prior to testing.

TABLE 3

| | Formulation (wt %) | | | | | | | | |
|---|---|---|---|---|---|---|---|---|---|
| Component | CF1 | CF2 | CF3 | CF4 | CF5 | F1 | F2 | F3 | F4 |
| Cocamidopropyl betaine¹ | 2.1 | 2.1 | 2.1 | 2.1 | 2.1 | 2.1 | 2.1 | 2.1 | 2.1 |
| C₈₋₁₄ alkyl polyglucoside² | 1.05 | 1.05 | 1.05 | 1.05 | 1.05 | 1.05 | 1.05 | 1.05 | 1.05 |
| Surfactant A³ | 9 | — | — | — | — | — | — | — | — |
| Surfactant B⁴ | — | 9 | — | — | — | — | — | — | — |
| Surfactant C⁵ | — | — | 9 | — | — | — | — | — | — |
| Surfactant D⁶ | — | — | — | 9 | — | — | — | — | — |
| Comp. Prod. Synthesis S5 | — | — | — | — | 9 | — | — | — | — |
| Inventive Prod. Synthesis S3 | — | — | — | — | — | 9 | — | 4.5 | 2.25 |
| Inventive Prod. Synthesis S4 | — | — | — | — | — | — | 9 | 4.5 | 6.25 |

¹Mackam C-37 from Solvay
²EcoSense ™ 3000 from The Dow Chemical Company
³Commercial 1-mole sodium laureth sulfate
⁴Commercial 2-mole sodium laureth sulfate
⁵Commercial 3-mole sodium laureth sulfate
⁶Commercial sodium lauryl sulfate

### Foam Performance

The foam performance of the personal care cleansing formulations of Comparative Examples CF1-CF5 and Examples F1-F2 was assessed by loading a 50 mL sample of a 3% formulation dilution in water into a Kruss Dynamic Foam Analyzer Instrument. Foam was generated with a 4,000 rpm mixing speed for 10 sec, with a 5 sec oscillation period. After mixing, foam height and bubble size were continuously monitored for five minutes. The results of the foam analyses are provided in TABLE 4.

TABLE 4

| | |
|---|---|
| Bubble radius (μm) | Foam height (mm) |

| Example | 30 sec | 300 sec | 30 sec | 300 sec |
|---|---|---|---|---|
| CF1 | 37 | 50 | 48.4 | 38.9 |
| CF2 | 40 | 54 | 44.0 | 37.4 |
| CF3 | 44 | 60 | 42.2 | 33.6 |
| CF4 | 37 | 49 | 50.9 | 40.0 |
| CF5 | 39 | 84 | 41.8 | 33.9 |
| F1 | 42 | 56 | 41.0 | 32.8 |
| F2 | 42 | 56 | 44.7 | 34.6 |

### Salt Thickening

The salt thickening of the personal care cleansing formulations of Comparative Examples CF1-CF5 and Examples F1-F4 was assessed by preparing dilutions of neat formulations in 1 mL vials on a 700 μL scale such that final formulations contained 10 wt % or 5 wt % surfactant actives with 0, 0.25, 0.5, 0.7, 1 and 1.5 wt % added sodium chloride salt. After dilutions and salt addition, the formulations were mixed by inverting and vortexing vials until solutions were uniform. Reported viscosities were obtained using a high-throughput methodology based on pressure measurements through pipette tips aspirating the samples using the Total Aspiration and Dispense Monitoring (TADM) system capability of a Hamilton liquid dispensing robot. Details of the method used can be found in ACS Comb. Sci. 2016, 18, 405-414. The results are provided in TABLE 5.

TABLE 5

| | Viscosity (cP) | | | | | |
|---|---|---|---|---|---|---|
| | wt % NaCl added | | | | | |
| Example | 0 | 0.25 | 0.5 | 0.75 | 1.0 | 1.5 |
| CF1 | 5.3 | 6.4 | 12.6 | 27.3 | 118.4 | 479.1 |
| CF2 | 4.9 | 4.5 | 5.7 | 7.2 | 12.2 | 58.9 |
| CF3 | 5.1 | 4.6 | 4.9 | 4.8 | 5.7 | 7.1 |
| CF4 | 5.1 | 5.3 | 8.9 | 16.6 | 79.8 | 511.0 |
| CF5 | 6.2 | 9.5 | 20.7 | 62.5 | 557.7 | 1,275.0 |
| F1 | 5.0 | 5.0 | 6.2 | 7.8 | 11.1 | 20.1 |
| F2 | 90.7 | 242.2 | 1,337.0 | 2,333.0 | >10,000 | >10,000 |
| F3 | 6.1 | 7.9 | 14.4 | 23.7 | 70.0 | 333.2 |
| F4 | 10.4 | 22.4 | 79.7 | 219.4 | 532 | 1,566 |

We claim:

1. An aqueous personal care rinse off composition, comprising:
a dermatologically acceptable aqueous vehicle; and
a dermatologically acceptable cleaning surfactant, wherein the dermatologically acceptable cleaning surfactant comprises an alcohol ethoxysulfate surfactant of formula I 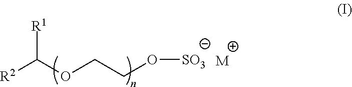
wherein each R¹ and R² is independently a C₁₋₁₆ alkyl group;
wherein the sum of the carbon atoms in R¹ and R² is 7 to 17;
wherein M⁺ is a cation balancing the negative charge of the —SO₃⁻ anion of formula I; and
wherein n is 1 in 95 to 100 mol % of the alcohol ethoxysulfate surfactant of formula I.

2. The aqueous personal care rinse off composition according to claim 1, wherein the alcohol ethoxysulfate surfactant of formula I contains <9 ppm of 1,4-dioxane.

3. The aqueous personal care rinse off composition according to claim 2, wherein the alcohol ethoxysulfate surfactant of formula I has elevated thermal stability (preferably, enhanced thermal stability).

4. The aqueous personal care rinse off composition according to claim 2, wherein the aqueous personal care rinse off composition contains <1 wt %, based on solids weight of the aqueous personal care rinse off composition, of an alcohol sulfate surfactant of formula II 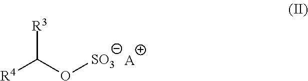
wherein each R³ and R⁴ is independently a C₁₋₁₆ alkyl group; wherein the sum of the carbon atoms is R³ and R⁴ is 7 to 17 and wherein A⁺ is a cation balancing the negative charge on the —SO₃⁻ anion in formula II.

5. The aqueous personal care rinse off composition according to claim 4, wherein the aqueous personal care rinse off composition is selected from the group consisting of a shampoo, a conditioning shampoo, a body wash formulation, an exfoliating body wash formulation, a facial wash formulation, an exfoliating facial wash formulation and a liquid hand soap formulation.

6. The aqueous personal care rinse off composition according to claim 5, wherein the dermatologically acceptable aqueous vehicle comprises water.

7. The aqueous personal care rinse off composition according to claim 6, wherein the dermatologically acceptable cleaning surfactant further comprises an additional surfactant selected from the group consisting of alkyl polyglucosides, alkyl poly pentosides, glycinates, betaines, taurates, glutamates, sarcosinates, isethionates, sulfoacetates, alaninates, amphoacetates, sulfonates, sulfates, succinates, sophorolipids, rhamnolipids and mixtures thereof; and wherein the weight ratio of alcohol ethoxysulfate surfactant of formula I to the additional surfactant in the aqueous personal care rinse off composition is 10:1 to 1:10.

8. The aqueous personal care rinse off composition according to claim 7, further comprising a thickening salt.

9. The aqueous personal care rinse off composition according to claim 7, wherein the additional surfactant includes a mixture of a betaine and an alkyl polyglucoside; wherein the thickening salt is sodium chloride and wherein the aqueous personal care rinse off composition comprises 0.1 to 20 wt %, based on weight of the aqueous personal care rinse off composition, of sodium chloride.

10. A method of cleaning at least one of mammalian skin and hair, comprising:
(a) applying an aqueous personal care rinse off composition according to claim 1 to the skin or hair of a mammal; and
(b) rinsing the aqueous personal care rinse off composition from the skin or hair with a rinse water.
